(19) United States
(12) Patent Application Publication
(10) Pub. No.: US 2016/0185793 A1
(43) Pub. Date: Jun. 30, 2016

(54) METHOD FOR THE PURIFICATION OF EPOTHILONES VIA CRYSTALLIZATION

(71) Applicant: SANDOZ AG, Basel (CH)

(72) Inventors: Mathias LUBBE, Dresden (DE); Christian SCHICKANEDER, Dresden (DE); Michael LIMMERT, Dresden (DE)

(21) Appl. No.: 14/911,404

(22) PCT Filed: Aug. 18, 2014
(86) PCT No.: PCT/EP2014/067529

§ 371 Date: Feb. 10, 2016

(30) Foreign Application Priority Data
Aug. 19, 2013 (EP) 13180859.4

(51) Int. Cl. C07D 493/04

(52) U.S. Cl. CPC C07D 493/04

(57) ABSTRACT

The present invention provides a new method of separating epothilones from one another that can be used on an industrial scale for the selective enrichment of epothilone and to provide a crystalline solid of enhanced purity, which can be used for the production of pharmaceutical preparations without using normal or reverse-phase chromatography or any energy input via distillation apparatus, wherein the new method comprises the steps:
a. dissolving a crude containing epothilones in an alkyl ester,
b. cooling the solution form a slurry,
c. isolating a crystalline solid from the slurry.

The present invention relates to a new method of separating epothilones from one another that can be used on an industrial scale and its usage in the production of pharmaceutical preparations.

Epothilones represent a relatively new class of microtubule-targeting agents that mimic the biological effects of taxanes. In contrast to taxanes, these macrolactone compounds are also active in vitro against multidrugresistant cancer cell lines. The six naturally occurring epothilones A to F have been identified and characterized to date (Altmann 2003, Mini Rev Med Chem. 2003, 3(2), 149-58.). For example, epothilones A and B, which nowadays find utility in the pharmaceutical field, having the structures:

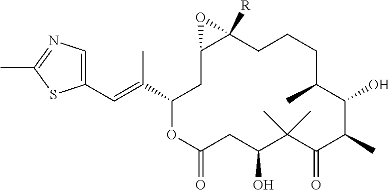

wherein R signifies hydrogen (epothilone A) or methyl (epothilone B).

Due to its biological activity, epothilone have been the target of many chemical total syntheses, which at the same time can provide a high degree of purity.

Danishefsky et al. (Angew. Chem. 1996, 108(23-24), 2976) discloses the first total synthesis of epothilone B, achieved via an extension Suzuki coupling reaction and a subsequent stereoselective epoxidation with dimethyldioxirane. Taylor and Chen (Org. Lett. 2001, 3(14), 2221-2224) disclose a total synthesis of epothilone B and D, wherein the route is highlighted by a final epoxidation of epothilone D yielding epothilone B in 65% yield, containing unconverted epothilone D.

Despite the success of epothilone total synthesis, these efforts are tedious, time-consuming, and expensive.

Due to their anti-fungal properties, epothilones were initially investigated as plant protective agents, which were originally obtained by fermentation as secondary metabolite of myxobacteria. Herein, the strain Sorangium (S.) cellulosum was found to produce and secreted the epothilone forms A and B.

WO 9310121 A1 discloses a method for cultivation of S. cellulosum in a fermentation medium containing carbon sources, nitrogen sources and mineral salts. During the fermentation process various synthesized epothilones are bound to an adsorber resin. The bound epothilones were separated by reverse-phase chromatography and crystallized in toluene/ethyl acetate or ethyl acetate.

When isolated in the pure form, epothilones A and B showed broad cytotoxic activity against eukaryotic cells and selectivity against breast and colon tumor cell lines.

US 2007/0122891 A1 discloses methods to improve the ratio of epothilone B to A produced by S. cellulosum, by adding a propionate as additive to the fermentation media. US 2007/0122891 A1 further discloses a process for the purification of epothilone B from the fermentation media, achieved by a combination of chromatography and crystallization or by crystallization only. The crystallization is carried out by extracting a resin, containing epothilones with a first distillative solvent, preferably ethyl acetate and swap it with a second solvent, characterized by a high boiling point, wherein the distillative solvent is distilled away to allow crystallization of epothilone B.

Disadvantageously, the high energy input during distillative processes easily results in decomposition of epothilone B, which carries a thermolabile epoxide ring structures.

EP 1 428 826 A2 discloses a method of separating epothilones, especially epothilone A and B from another, which is characterized by chromatography on a reversed-phase column using an acetonitrile/water mixture as an eluent.

Reverse-phase chromatography has enjoyed widespread acceptance as a rapid, moderate purification technique. However it suffers from the great disadvantages in consumption high amounts of solvents and high costs for equipment. Furthermore, reverse-phase chromatography is very difficult to handle and unsuitable for large-scale production.

The invention has the object of finding a method for the purification of epothilones, especially epothilone B for large-scale industrial application, which has significant potential to save primary energy and reduce emissions. In particular the method shall reduce the amounts of solvents needed for purification.

The objective of the invention is solved by a method of separating epothilones from another, preferably from a mixture containing epothilone B, with the following steps:
a. dissolving a crude containing epothilones in an alkyl ester,
b. cooling the solution to −15° C. to 15° C. to form a slurry,
c. isolating a crystalline solid, preferably containing epothilone B, from the said slurry.

Advantageously the invention enables a purification of epothilones from impurities via crystallization without using normal or reverse-phase chromatography or any energy input via distillation apparatus.

The general terms used hereinabove and herein below preferably have the meanings given herein below:

Where reference is made hereinabove and herein below to documents, these are incorporated insofar as is necessary.

The crude containing epothilones is preferably obtained as fermentation product and/or by chemical synthesis. A preferred fermentation is performed in S. cellulosum, wherein the cultivation conditions of the recombinantly produced microorganisms are known by those skilled in the art.

A crude containing epothilones can also be obtained by a chemical total synthesis, such as those disclosed by Meng et al. (I. Am. Chem. Soc. 1996, 119(42), 10073-10092); Nicolaou et al. (J. Am. Chem. Soc. 1997, 119(34), 7974-7991) and Schinzer et al. (Chem. Eur. J. 1999, 5(9), 2483-2491).

The epothilones occurring in a crude are preferably epothilone A and/or B, but also other epothilones, for example epothilones C and D named in International Application WO 97/19086 and WO 98/22461, epothilones E and F named in WO 98/22461, and further epothilones obtainable from corresponding microorganisms.

In some embodiments, the epothilones are selected from ixabepilone, epothilone B, epothilone D, BMS310705, dehydelone and ZK-EPO.

As used herein, the term “crude containing epothilones” refers to a mixture containing epothilone B and at least another epothilone selected from epothilones A, C, D, E, F, ixabepilone, BMS310705, dehydelone and ZK-EPO, which is in a substantially unrefined state. The crude containing epothilones is preferably obtained as fermentation product or alternatively by chemical synthesis or a combination thereof.

Epothilones according to the invention are macrolactones characterized by a relatively low content of functional groups.

Preferably in the crude the amount of epothilone B is in a molar ratio to the amount of pothilone A in the range of 15:1 to 1:2, more preferably of 10:1 to 1:1.

A crude containing epothilones can also contain impurities such as particulate materials, inorganic nutrients, sugars, organic acids, and amino acids. Preferably, the amount of impurities in a crude is less than 5.0 percent by weight, more preferably less than 3.0 percent by weight based on the total weight of the crude.

However, a purification process according to the invention is suitable to separate impurities, so that in a crystalline solid the amount of impurities is less than 2.0 percent by weight, more preferably less than 1.0 percent by weight based on the total weight of the crystalline solid.

As used herein, the solvent is an alkyl ester, preferably an alkyl acetates, wherein the alkyl acetates are preferably selected form the group consisting methyl acetate, ethyl acetate, isopropyl acetate, n-propyl acetate, n-butyl acetate, isobutyl acetate, sec-butyl acetate and tert-butyl acetate or a mixture of any of at least two of the listed esters. Most preferably, the alkyl acetate is methyl acetate.

An alkyl ester according to the present invention is characterized in a low melting point, preferably below −40° C., more preferably below −60° C.

Preferably, the crude is dissolved in the alkyl ester to a mass concentration of between 10 and 100% (w/v), most preferably of between 12.5 and 75% (w/v), most preferably of between 15 and 50% (w/v) while the solutions are maintained at ambient temperature, preferably about 25±10° C.

Mass concentration is defined as the weight of the crude (w) per volume of the alkyl ester (v). Obviously, a large number of methods is available that enable a person of ordinary skill in the art to determine the mass concentration.

As used herein, the term “ambient temperature” refers to a temperature between 10° C. to 35° C., more preferably 15° C. to 30° C.

After dissolving a crude containing epothilones in an alkyl ester, the solution is cooled to −15 to 15° C., preferably to −10 to 10° C., more preferably to −10 to 5° C. The resulting solution (including the alkyl ester and the crude) exceeds the solubility limit of an epothilone thereby causing the epothilone to crystallize from the solution. Preferably epothilone B crystallizes from the solution, while other epothilones that may be present in the solution remain in the solution below their solubility limit.

To support ordered crystallization, preferably cooling the solution containing epothilones takes place via a cooling temperature profile (e.g. stepwise cooling profile, controlled cooling profile) or cooling temperature gradient (linear cooling method). Preferably the temperature of the solvent is gradually reduced at a cooling rate of 10 Kelvin per minute, more preferably 5 Kelvin per minute, most preferably 2 Kelvin per minute.

Optionally, the solution is seeded with appropriate epothilone crystal nucleuses in a sufficient quantity, preferably less than 2.0 percent by weight, more preferably less than 1.0 percent by weight based on the total weight of the solution in order to promote the crystallization process and to enhance purity of the crystalline solid.

An appropriate epothilone crystal nucleus also called seed crystal is a small piece of single-crystal/polycrystal epothilone from which a large crystal of epothilone is to be grown, preferably with the same crystal modification.

Advantageously, seeding with appropriate epothilone crystal nucleuses can support epitaxial growth of single-crystal, which comprises epothilone with a defined crystal modification.

A single-crystal is a highly ordered crystal, preferably characterized by only one crystal modification of an epothilone derivate.

Crystallization of the epothilone may be conducted using either a batch crystallization process or a continuous crystallization process.

In a batch process, the starting solution is prepared by dissolving a crude in an alkyl ester in a single vessel where crystallization occurs, when cooling the solution to a temperature between −15 and 15° C. After the crystals are isolated the process can be repeated.

Optionally a batch process according to the invention is carried out as a multistage crystallization process, wherein after each crystallization cycle a recrystallization of the impure crystalline solid, preferably after removing solvent containing impurities takes place by repeating the steps of dissolution in a fresh solvent, cooling and isolation, until a sufficient purity of the crystalline solid is reached.

In a continuous crystallization process, a solution is also prepared by dissolving a crude in an alkyl ester in a single container. The resulting solution is pumped continuously into a crystallization vessel, wherein at the same time, the temperature is decreased locally in the crystallization vessel that will result in the desired final temperature range giving hereinabove. Crystallization occurs in this vessel, which is typically at a higher rate than in a batch vessel due to the continual presence of seed crystals. The product slurry is continuously pumped out of the vessel, thus maintaining a constant volume in the crystallizer.

In both embodiments, the isolation of the crystalline solid is preferably carried out via filtration, preferably using a Büchner funnel, or by centrifugation, followed by a drying procedure to provide a final product.

Advantageously, solvents used in the process may be recovered by distillation. Dissolved epothilones in the supernatant can be recovered and/or either recycled.

The present invention further comprises a multistage crystallization process based on a plurality of cascaded crystallization cycles, wherein the basic three-step method according to the invention is repeated comprising:
1. providing a crystalline solid comprising epothilones, especially epothilone B and epothilone A in a molar ratio between 5:1 and 60:1, more preferably between 10:1 and 50:1, most preferably between 15:1 and 45:1, after a first run of the crystallization process comprising:
   a. dissolving a crude containing epothilones in an alkyl ester,
   b. cooling the solution to −15° C. to 15° C. to form a slurry,
   c. isolating a crystalline solid, preferably containing epothilone B, from the said slurry,
2. dissolving the crystalline solid obtained after the first run of the crystallization process in an alkyl ester, preferably in fresh alkyl ester, in accordance to process step a) of the present invention,
3. cooling the solution to a temperature ranging between −15 to 15° C., preferably to −10 to 10° C., more preferably to −10 to 5° C.,
4. isolating a high purity crystalline solid comprising epothilones, especially epothilone B and epothilone A,

wherein in a high purity crystalline solid the amount of epothilone B is increased as compared to the amount of epothilone B in the crystalline solid obtained from a previous run of the crystallization process, preferably by a factor of ranging between 1.1 to 2.0,

wherein the crystallization process according to steps 2) to 4) is repeated with the obtained purity crystalline solid for at least 1 time, more preferably at least 2 times, most preferably at least 3 times, but can be repeated unlimited number of times,

that the molar ratio of epothilone B and epothilone A is increased stepwise up to a final molar ratio between 100:1 and 175:1, more preferably between 110:1 and 165:1, and

wherein the total recovery of epothilone B after completion the multistage crystallization process is preferably in the range between 50 to 70%, more preferably between 55 to 65% by weight.

Advantageously, a multistage crystallization process can be used for the selective enrichment of epothilone, especially epothilone B in a crystalline solid. It is also advantageous that each repetition of the crystallization process (recrystallization) according to a multistage crystallization process leads to decreased impurity in the obtained crystalline solid.

Preferably, in accordance with the present invention, the crystallization and the subsequent recrystallizations are designed as a multistage crystallization process, wherein the method according to the invention is repeated in a series of vessels. Preferably a multistage crystallization process including introducing an impure crude containing epothilones into the first vessel and moving the crystalline solid progressively to the last vessel, wherein solvent containing impurities are removed from the crystalline solid after the first and each subsequent crystallization cycle and fresh alkyl ester as solvent is introduced to each vessel dissolving the impure crystalline solid.

Preferably the isolated crystalline solid is washed with the alkyl ester and dried, more preferably dried only. Advantageously, the processing is performed so that volatile solvent residues are removed to yield a high-quality epothilone.

Preferably the crystalline solid is dried by evaporation in a temperature range between 0 to 70° C., more preferably between 5 to 60° C., most preferably between 10 to 50° C. at a pressure of 1 to 1000 mbar, more preferably of 1 to 600 mbar, most preferably of 1 to 400 mbar.

The method of the invention is used to produce a purified crystalline solid comprising epothilones, especially epothilone B and epothilone A in a molar ratio between 5:1 and 60:1, more preferably between 10:1 and 50:1, most preferably between 15:1 and 45:1. In a purified crystalline solid the molar ratio between epothilone B and epothilone A is at least twice, preferably at least three times, most preferably at least four times as high as in the crude.

The purified epothilones of the invention can be used as an active ingredient for the preparation of pharmaceutical composition.

The following examples serve to explain the present invention in more detail without representing a restriction of the scope of protection defined in the Claims.

Caution: When handling compounds, appropriate protective measures must be taken, where necessary, in view of their high toxicity.

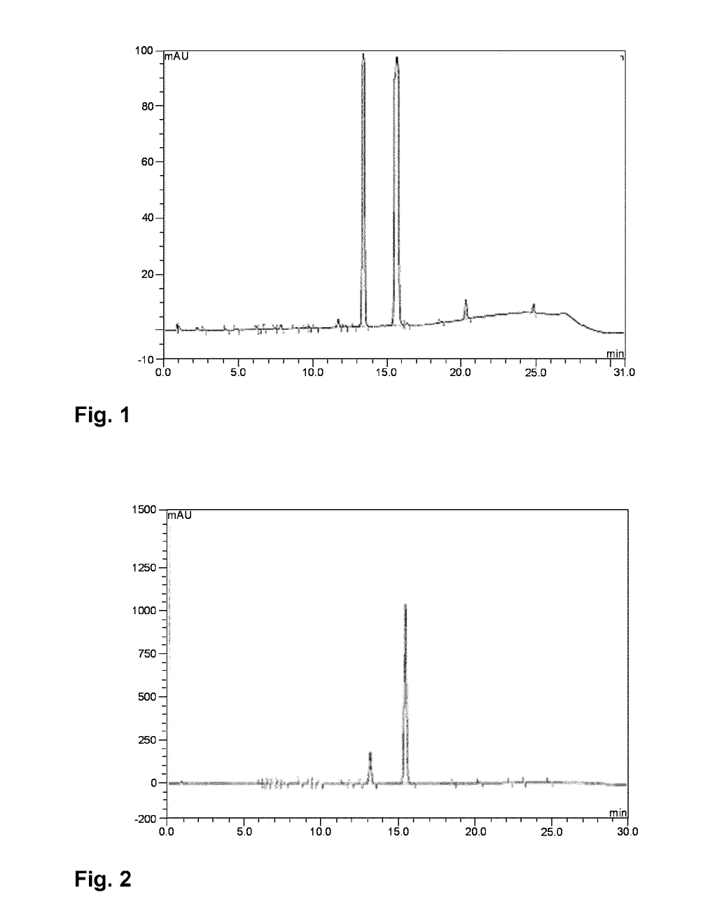

FIG. 1: Chromatogram of epothilone crude as starting material.

FIG. 2: Chromatogram of epothilone crude (A/B=1:6.1) as starting material.

### EXAMPLE 1

### Crystallization in Methyl Acetate

In a first step a defined mass of a crude (w in [g]) containing epothilone B and epothilone A in a molar ratio of 1 to 6.1 is placed in a flask and dissolved in a volume of methyl acetate (v in [mL]), while stirring at ambient temperature. The mixture is stirred at room temperature (22° C.) for 24 h.

When the mixture is cooled in an ice bath, it is stirred at 0° C. over a period of 9 h while precipitation occurs. The crystalline solid is isolated via filtration with a funnel and washed with methyl acetate at 0° C. The crystalline solid is dried by evaporation for 16 h, 20 mbar at 40° C.

| | mass of | molar | Volume of | | molar ratio |
|---|---|---|---|---|---|
| | epothilone | ratio of | methyl | Yield | between |
| | crude w in | epothilone | acetate v in | w/w | Epothilone |
| Nr. | [g] | B/A | [mL] | [%] | B/A |
| 1 | 0.5 | 6.1 | 5.0 | 78 | 31.4 |
| 2 | 40.0 | 6.1 | 200.0 | 79 | 25.7 |
| 3 | 5.0 | 6.1 | 35.0 | 73 | 31.5 |

### EXAMPLE 2

### Crystallization in Methyl Acetate

In a first step 5.0 g of an epothilone crude containing epothilone A and epothilone B in a molar ratio of 6.0 is suspended in 25 mL of methyl acetate. The mixture is stirred at 21° C. for 8 h. Afterwards the mixture is cooled to 2° C. and stirred at this temperature for 8 h while precipitation occurs. The crystalline solid is isolated via filtration with a funnel and washed with 5 mL of methyl acetate at 2° C. After drying, 3.8 g of a white crystalline solid is isolated, containing epothilone B and epothilone A in a molar ratio of 28.0 to 1.

### EXAMPLE 3

### Continuous Multistage Crystallization in Methyl Acetate

30.0 g of a crude containing epothilone A and B in a molar ratio of 1 to 6.0 were suspended in 165 mL of methyl acetate. The mixture was stirred at 22° C. for 9 h, cooled to 0° C. and stirred at this temperature for 9 h. The crystalline solid was isolated by filtration of the cold mixture and washed with 30 mL of cold (0 to 5° C.) methyl acetate to yield 27.3 g of moist epothilone crystals (HPLC: ratio of epothilone A/B=1:25).

In a second cycle 27.0 g of the moist epothilone crystals (A/B=1:25) were suspended in 148.5 mL of methyl acetate. The mixture was stirred at 22° C. for 9 h, cooled to 1° C. and stirred at this temperature for 9 h. The crystalline solid was isolated by filtration of the cold mixture and washed with 27 mL of cold (0 to 5° C.) methyl acetate to yield 25.5 g of moist epothilone B crystals (HPLC: ratio of epothilone A/B=1:45).

In a third cycle 25.2 g of the previously obtained moist epothilone B crystals (A/B=1:45) were suspended in 138.6 mL of methyl acetate. The mixture was stirred at 22° C. for 9 h, cooled to 0 to 5° C. and stirred at this temperature for 9 h. The crastalline solid was isolated by filtration of the cold mixture and washed with 25.2 mL of cold (0 to 5° C.) methyl acetate to yield 20.7 g of moist epothilone B crystals (HPLC: ratio of epothilone A/B=1:77).

In a fourth cycle 20.4 g of the previously obtained moist epothilone B crystals (A/B=1:77) were suspended in 112.2 mL of methyl acetate. The mixture was stirred at 22° C. for 9 h, cooled to 4° C. and stirred at this temperature for 9 h. The solid was isolated by filtration of the cold mixture and washed with 20.4 mL of cold (0 to 5° C.) methyl acetate, wherein 17.5 g of a moist epothilone B product was yield. The moist epothilone B product was dried at 40° C. at a pressure of 20 mbar for 10 h to yield 13.6 g of a crystalline epothilone B solid (HPLC: ratio of epothilone A/B=1:125).

In a fifth cycle 13.3 g of the previously obtained epothilone B crystals (A/B=1:125) were suspended in 73.2 mL of methyl acetate. The mixture was stirred at 22° C. for 9 h, cooled to 3° C. and stirred at this temperature for 9 h. The solid was isolated by filtration of the cold mixture and washed with 13.3 mL of cold (0 to 5° C.) methyl acetate. The moist product (12.4 g) was dried at 40° C. at a pressure of 20 mbar for 34 h to yield 11.1 g of an epothilone B crystal (HPLC: ratio of epothilone A/B=1:154) with a total recovery of epothilone B of 43.2%.

1. A method of separating an epothilone from a mixture containing epothilone B, comprising the steps:
a. dissolving a crude containing epothilones in an alkyl ester,
b. cooling the solution to −15° C. to 15° C. to form a slurry, and
c. isolating a crystalline solid containing epothilone B from the said slurry.

2. A method according to claim 1, wherein the epothilones are epothilone B and epothilone A.

3. A method according to claim 2, wherein in the crude the amount of epothilone B is in a ratio to the amount of epothilone A in the range of 15:1 to 1:2.

4. A method according to claim 1, wherein the crude is dissolved in the alkyl ester to a mass concentration of between 10 and 100%.

5. A method according to claim 1, wherein the alkyl ester is an alkyl acetate.

6. A method according to claim 1, wherein cooling of the solution is assured via a cooling temperature profile or a gradient.

7. A method according to claim 5, wherein the alkyl acetate is methyl acetate.

8. A method according to claim 1, wherein the solution is seeded with ephothilone crystals.

9. A method according to claim 1, wherein the steps of the method are carried out as a multistage crystallization process.

10. A method according to claim 1, wherein the crystalline solid is dried by evaporation.

11. A method according to claim 1, wherein the crystalline solid contains epothilone B and epothilone A in a molar ratio between 5:1 to 60:1.

12. The method according to claim 1 wherein the crystalline solid comprises epothilone B and epothilone A in a molar ratio of 5:1 to 60:1.

13. A pharmaceutical preparation comprising an isolated crystalline solid containing epothilone B obtained by the process of claim 1.
